(19) United States
(12) Patent Application Publication
(10) Pub. No.: US 2007/0071825 A1
(43) Pub. Date: Mar. 29, 2007

(54) Device and method for making particles

(76) Inventors: Catherine Curdy, Martigny (CH); Betrand Ducrey, Martigny (CH); Frederic Heimgartner, Villeneuve (CH); Francois Pfefferle, Grimisuat (CH)

Correspondence Address: NIXON & VANDERHYE, PC, 901 NORTH GLEBE ROAD, 11TH FLOOR, ARLINGTON, VA, 22203, US

(21) Appl. No.: 10/574,003

(22) PCT Filed: Sep. 28, 2004
(86) PCT No.: PCT/IB04/03151

§ 371 Date: Sep. 29, 2006

(30) Foreign Application Priority Data
Oct. 1, 2003 (CH) 1664/03

(51) Int. Cl. A61K 9/14; B29B 9/00; B05C 11/00

(52) U.S. Cl. 424/489; 264/5; 118/600; 977/700; 977/906

(57) ABSTRACT

Device for continuously producing particles, consisting of a homogenising compartment (1) including at least one inlet (2) for feeding in an organic phase, an inlet (3) for feeding in an aqueous phase, a mixing system (4) and an outlet (5).

The invention relates to a device for the manufacture of microparticles or nanoparticles and to its use in a process for the manufacture of said particles.

It is known to produce microparticles and nanoparticles by the use of processes in which an organic phase is mixed with an aqueous phase. The industrial preparation of nanoparticles and microparticles presents a problem due to their small size. Various processes and uses of devices for the preparation of microparticles and nanoparticles are described in the literature. The use is known in particular of techniques such as nebulization in a stream of hot air (spray drying) or cold air (spray cooling), phase separation, emulsion-solvent extraction, emulsion-solvent evaporation or supercritical fluids.

Furthermore, with the techniques currently used, it is in particular difficult to obtain particles which are uniform in shape and homogeneous in size. Furthermore, difficulties during the stage of isolation of such particles, in particular by filtration or by sieving, do not make it possible to optimize the manufacturing output.

EP 0 471 036 discloses a process for the manufacture of nanoparticles and microparticles in which, in a homogenizer of Silverson type, an organic phase is dispersed in a medium saturated with solvent identical to that present in said organic phase, so as to form a first oil-in-water emulsion. This emulsion, composed of microdrops, is transferred as rapidly as possible into a medium which makes it possible to extract 20 to 30% of the solvent present in the microdrops. This second stage makes it possible to obtain hardened microparticles and nanoparticles. Furthermore, the problem encountered with this process is that it is necessary to adapt the equipment according to the amounts of the starting organic phase.

WO 98/35654 discloses a continuous process for the manufacture of microparticles. With the device used, the process does not make it possible to obtain particles of definite size and of uniform shape. This is because the positioning of the inlets of the phases and that of the outlet of the homogenization compartment are such that a volume of air partially occupies the homogenization compartment all along the homogenization phase and, for this reason, turbulence is created in the compartment, resulting in the formation of particles of nonuniform shape.

WO 03/033097 relates to the use of a rotor/stator device for the manufacture of fine particles by a precipitation or crystallization method. In the device of this prior art, use is not made of a hollow tube and even less of an inlet means which is perforated for better diffusion of the phases in a homogenization chamber. Furthermore, the rotor used is a rotor with walls having a “hopper”-type structure. Moreover, the phases involved are subjected to stirring forces during their mixing.

Finally, it should be noted that the positioning of the outlet is such that a vacuum is inevitably formed, which does not promote the formation of small particles of uniform shape.

In the device according to the present invention, due to the perpendicular positioning of the teeth of the rotor/stator, two phases pass through the teeth and these are the shear forces which contribute to good homogenization of the mixture and the preparation of uniform particles which are small in size.

The problems encountered in the past could be solved by the device according to the invention and its use in a continuous process for the manufacture of nanoparticles or microparticles according to the invention.

One of the aims of the present invention is thus to provide a device which makes possible control of the size of the finished product and which thus makes it possible to continuously produce both nanoparticles and microparticles of uniform shape.

Another aim of the present invention is to optimize a process for the manufacture of nanoparticles or microparticles by employing the device according to the invention so as to rapidly and continuously produce solid and separate nanoparticles or microparticles of uniform shape.

One of the objects of the present invention is a device comprising a homogenization compartment, itself comprising a filling system, a rotor/stator system and an outlet means, for the continuous manufacture of microparticles or nanoparticles from at least one aqueous phase and one organic phase.

Another object of the present invention is a process for the manufacture of microparticles or nanoparticles employing said device.

Furthermore, in a continuation of the description, the expression “a rotor/stator system” will be used to denote a system composed of a stationary component, “the stator”, into which is inserted a moving component, “the rotor”. In a specific form of the invention, “the rotor” and “the stator” are equipped with teeth which make possible, by rotation, the mixing of various substances and their homogenization.

The expression “teeth of the rotor” will be used to denote the projecting parts of the rotor and stator.

In the continuation of the description, the expression “homogenization compartment” will be used to denote the closed chamber in which the phases are brought into contact and are homogeneously mixed.

In the continuation of the description, the expression “perforations” will be used to denote holes with a size of less than or equal to 1 mm.

In the continuation of the description, the expression “hollow tube” will be used to denote a pipe which allows substances to pass.

In the continuation of the text, the expression “active substance” will be used to denote a substance having at least one pharmaceutical characteristic.

In the continuation of the text, the expression “solvent” will be used to denote the organic medium in which one or more polymers is/are dissolved.

In the continuation of the text, the expression “polymer” will be used to denote the matrix composed of polymerized units acting as agent controlling the release of the active substances.

The expression “storage receptacle” will be used to denote the receptacle placed at the outlet of the homogenization compartment for the purpose of collecting a sufficient volume of particle suspension allowing the priming of a pump for the filtration or ultrafiltration of the particles.

The expression “aqueous phase” will be used to denote the external phase composed at least of water and a surfactant which makes possible the extraction of the organic solvent and the hardening of the particles.

The expression “surfactant” will be used to denote the substance added to the aqueous phase which makes it possible to stabilize the emulsion.

The expression “organic phase” will be used to denote the solution or the suspension or the emulsion comprising at least one polymer and one active substance.

The present invention relates to a device for the continuous manufacture of microparticles or nanoparticles from at least one aqueous phase and one organic phase composed of a homogenization compartment (1) comprising at least one inlet (2) for delivering the organic phase, one inlet (3) for delivering the aqueous phase, one mixing system (4) and one outlet (5), characterized in that
a) the inlet (2) is a hollow tube for delivering the organic phase and is positioned coaxially with the axis of said mixing system (4),
b) the tip (6) of said hollow tube is in a volume (A) delimited by the mixing system (4) in the homogenization compartment (1),
c) the tip (7) of the inlet (3) is in the volume (B) delimited between the wall (8) of the homogenization compartment (1) and the end (9) of the mixing system (4), and
d) the outlet (5) is in the top wall of the homogenization compartment.

In the device according to the present invention, the inlet (2) and the outlet (5) are positioned so that it is possible to prevent an excess entry of air into the homogenization compartment in order to prevent the formation of misshapen particles.

The inlet (2) is positioned coaxially with the axis of the mixing system (4), i.e. in the axis of said system, and the outlet (5) is in the top wall of the homogenization compartment (1).

In the device according to the present invention, the inlet (2) is a hollow tube for delivering the organic phase and the inlet (3) for delivering the aqueous phase are positioned so that these two phases are delivered simultaneously and homogeneously to the homogenization compartment (1).

Moreover, in order to promote good dispersing of the organic phase in the aqueous phase, the tip (6) of said hollow tube is in a volume (A) delimited by the mixing system (4) in the homogenization compartment (1) and the tip (7) of said inlet (3) is in a volume (B) delimited between the wall (8) of the homogenization compartment (1) and the end (9) of the mixing system (4).

Preferably, in the device according to the invention, the hollow tube is or is not closed at its tip (6) and exhibits perforations (10) so as to promote fine dispersing of the organic phase in the aqueous phase in the homogenization compartment (1).

The perforations (10) occur on the final part of the hollow tube entering the volume (A). They may be in one or more rows or be random.

In one embodiment of the device according to the invention, the number of perforations is a minimum of 1 to 5. In an advantageous embodiment, the number is from 1 to 10 and, in an even more advantageous embodiment, the number is from 1 to 20.

The perforations (10) can be obtained mechanically by perforation of the wall of the hollow tube using a microdrill or a laser, for example.

It is also possible to use a hollow tube having a final part, present in the volume (A), made of a material such as, for example, sintered glass or metal mesh. There is thus present a hollow tube having a final part possessing a multitude of perforations (10) which can be less than 0.01 mm in size.

The perforations (10) can be from 0.01 mm to 1 mm. Preferably, the perforations (10) are from 0.01 mm to 0.9 mm and more preferably still from 0.01 mm to 0.7 mm. The choice of the size of the perforations also makes it possible to optimize the dispersing of the organic phase in the aqueous phase in the homogenization compartment (1) but, in particular, to optimize the exactness of the size desired for the nanoparticles or the microparticles.

In the device according to the invention, the tangential velocity of the mixing system (4) is from 1.5 m/s to 50 m/s and preferably from 2.5 m/s to 41 m/s.

In one embodiment of the invention, the mixing system (4) is a rotor (11)/stator (12).

The rotor (11)/stator (12) system can comprise at least one row of teeth (13) and the spacing (14) between the teeth (13) can be from 1 to 4 mm. The smaller the spacing, the easier it is to produce particles which are small in size. Conversely, the greater the spacing, the easier it is to produce particles which are larger in size.

Preferably, the dimensions of the rotor (11)/stator (12) system are such that said system occupies 4% to 40% of the volume of the homogenization compartment (1).

Another subject matter of the present invention is a continuous process for the manufacture of microparticles or nanoparticles employing the device according to the invention.

Said process is such that an organic phase comprising at least one active substance, one polymer and one solvent and an aqueous phase comprising at least one surfactant are simultaneously delivered, via the inlet (2), which is a hollow tube, and via the inlet (3) respectively, to the homogenization compartment (1) in which the mixing system (4) has a tangential velocity of 1.5 m/s to 50 m/s, making possible simultaneously the formation of an emulsion of said phases and the extraction of the solvent present in the organic phase, so as to obtain a suspension of particles from which the nanoparticles or microparticles are isolated.

Preferably, in the process according to the invention employing said device, the tangential velocity of the mixing system (4) is from 1.5 m/s to 50 m/s and at least from 2.5 m/s to 41 m/s.

Preferably, in the process according to the invention, the mixing system 4 is a rotor (11)/stator (12) system.

In a preferred form of the process according to the invention, the organic phase is delivered via the hollow tube which is or is not closed at its tip (6) and which exhibits perforations (10), so as to radially disperse said phase in the aqueous phase in the homogenization compartment (1).

In order to isolate the nanoparticles or microparticles from the particle suspension, it is possible to discharge said suspension via the outlet (5) of the homogenization compartment (1) and then to carry out a direct or tangential filtration. It is also possible to carry out a simple or forced settling using a device of continuous centrifuging or drying machine type, after discharge of said suspension via the outlet (5) of the homogenization compartment (1).

In one embodiment of the process according to the invention for the manufacture of nanoparticles, the nanoparticles are isolated from the particle suspension by discharging said suspension via outlet (5) of the homogenization compartment (1) into a storage receptacle and by then subjecting said suspension to continuous ultrafiltration.

In another embodiment of the process according to the invention for the manufacture of microparticles, the microparticles are isolated from the particle suspension by discharging said suspension via the outlet (5) of the homogenization compartment (1) into a storage receptacle and by then subjecting said suspension to continuous filtration.

In the process according to the invention, the organic phase can comprise 1 to 30% of polymer in at least one solvent but at least 2 to 25% and in all cases 5 to 20%.

The polymer/active substance mixture present in the organic phase can comprise 0.1 to 50% of active substance but at least 0.5 to 40% of active substance and in all cases 1 to 30% of active substance.

In the process according to the present invention employing said device, the organic phase can be in the solution, emulsion or suspension form.

The organic phase is in the solution form when the active substance is dissolved with the other compounds of the organic phase.

The organic phase is in the emulsion form when the active substance is dissolved in water and then emulsified with the other compounds of the organic phase.

Finally, the organic phase is in the suspension form when the active substance is not dissolved and when it occurs in the form dispersed in the organic phase.

The water-soluble active substance can in particular be a peptide, a polypeptide, a protein or respectively a salt which is acceptable from a pharmaceutical viewpoint.

According to the present invention, the active substance can be gonadorelin (LHRH) or one of its derivatives (agonists and antagonists), thyrotropin (TSH), protirelin (TRH), follicle stimulating hormone (FSH), parathyrin (PTH), insulin and other hypoglycemic peptides, C-peptide, exenatide analogs, analogs of GLP-1 and other antiobesity peptides, antagonists of the TCR receptor of lymphocytes, somatostatin or one of its derivatives, corticotrophin (ACTH), a growth hormone (GH), somatorelin (GHRH), growth hormone releasing peptide (GHRP), calcitonin, endorphin, an interferon, an interleukin, tumor necrosis factor (TNF), erythropoietin (EPO), a colony stimulating factor (G-CSF, GM-CSF, M-CSF), a nerve growth factor (NGF), a somatomedin (IGF), amylin and its synthetic analogs, bone morphogenic protein (BMP), serotonin, GABA, superoxide dismutase, an immunomodulator (EGF, LPS), an anticancer, such as actinomycin D, bleomycin, busulfan, carboplatin, cisplatin, oxaliplatin, carmustine, chlorambucil, cladribine, cyclophosphamide, cytarabine, dacarbazine, daunorubicin, doxorubicin, estramustine, etoposide, floxuridine, fludarabine, fluorouracil, hexamethylmelamine, hydroxyurea, idarubicin, ifosfamide, asparaginase, lomustine, mechlorethamine, melphalan, mercaptopurine, methotrexate, mithramycin, mitomycin C, mitotane, mitoxantrone, pentostatin, procarbazine, streptozocin, teniposide, thioguanine, thiopeta, vinblastine, vincristine and the like; an antiviral; an analgesic, such as pethidine hydrochloride, levorphanol tartrate, morphine hydrochloride or oxymorphone; a narcotic antagonist, such as naloxone, naltrexone or others; a local anesthetic, such as lidocaine, xylocaine and the like; cyclosporine and derivatives; an antiepileptic; an antidepressant; an anticoagulant, such as natural or synthetic heparin; an elastase inhibitor, such as EPI-hNE, particularly EPI-hNE-4; a substance for the treatment of retinal degeneration, such as a steroid or another peptide substance; an antifungal; a bone resorption inhibitor, such as a bisphosphonate, alendronate and the like; an antigen for bacteria, a virus; an antidiabetic, such as glizipide; an enzyme; a nucleic acid; an antipsychotic neuroleptic, such as olanzapine, risperidone and the like; an α-reductase inhibitor, such as finasteride or dutasteride; an aromatase inhibitor, such as anastrozole and exemestane; a hormone, such as thyroxins or estrogens; a hormone therapy substance, such as tamoxifen and 4-OH tamoxifen; a vitamin; huperzine and its derivatives.

The active substance can be a peptide salt. It can be a mono-, di- or trisalt. According to the present invention, the peptide salt can be a salt formed with an inorganic acid, such as hydrochloric acid, sulfuric acid or nitric acid, for example. It can also be a salt formed with an organic acid, such as, for example, carbonic acid, bicarbonic acid, succinic acid, acetic acid, propionic acid or trifluoroacetic acid. Preferably, the peptide salt is a salt formed with an organic acid and, in an advantageously preferred way, the organic acid is acetic acid or pamoic acid.

The preferred active substance is olanzapine, alendronate, tamoxifen, 4-OH tamoxifen, and derivatives, LHRH derivatives, in particular triptorelin pamoate, somatostatin derivatives, in particular vapreotide pamoate, natural or synthetic heparin, neuroleptics, PTH, insulin and other hypoglycemic peptides, C-peptide, exenatide and its analogs, GLP-1 and its analogs, cyclosporine and its derivatives, calcitonin, interferons, interleukins, EPO, CSF, oxaliplatin, antidiabetics, such as glizipide, α-reductase inhibitors, thyroxin, estrogens, huperzine and its derivatives.

According to the present invention, the polymer used is preferably a biodegradable or biocompatible polymer selected from polylactic acids, polyglycolic acids, copolymers of lactic and glycolic acids, copolymers of lactic acid and caprolactone or other biodegradable polymers, such as other aliphatic polymers, polycitric acid, polymalic acid, polysuccinates, poly(butyl succinate)s, polyfumarates, polyhydroxybutyrates, polycaprolactones, polycabonates, polyesteramides, polyanhydrides, poly(amino acid)s, polyorthoesters or their copolymers with PEG, poly(alkyl cyanoacrylate)s, polyetheresters, polydioxanones, copolymers with polyethylene glycol, such as, for example, the PBS-PEG coblocks disclosed in WO 99/55760, the PLA-PEG coblocks disclosed in U.S. Pat. No. 5,766,635, or PLGA-PEG coblocks, polyurethanes which are biodegradable and polyphosphazenes or their copolymers with PEG.

Appropriate nonbiodegradable polymers are polyacrylic acid, polymethacrylic acid, copolymers of acrylic acid and methacrylic acid, ethylcellulose, acetylcellulose, nitrocellulose, and the like. These polymers can be homopolymers or copolymers of two monomers or more or also blends of polymers.

Preferably, the polymer is selected from the group consisting of copolymers of lactic acid and glycolic acid, polylactic acid, copolymers of polylactic acid and caprolactone, copolymers of polyethylene glycol or polypropylene glycol with other groups, such as PLGA-PEG coblocks, PLA-PEG or PBS-PEG, polyorthoesters and polyphosphazenes and their copolymers with PEG.

According to the present invention, the organic solvent used is chosen from water-immiscible or virtually water-immiscible solvents, such as esters, for example ethyl acetate, or butyl acetate, halogenated hydrocarbons, such as dichloromethane, chloroform, chloroethane, dichloroethane or trichloroethane, ethers, such as ethyl ether or isopropyl ether, aromatic hydrocarbons, such as toluene or xylene, carbonates, such as diethyl carbonate, or the like.

Preferably, the solvent used is an ester or a halogenated hydrocarbon.

Preferably, the solvent used is ethyl acetate.

Water-miscible solvents, such as ethanol, dimethylformamide, dimethyl sulfoxide, substituted pyrrolidones, such as N-methyl pyrrolidone, or propylene glycol, can be added to the water-immiscible solvents.

In one embodiment of the process according to the invention, use may be made of the solvents mentioned above, alone or as mixtures.

In a preferred form of the process according to the present invention, the organic phase comprises at least
as active substance, olanzapine, alendronate, tamoxifen, 4-OH tamoxifen, and derivatives, LHRH derivatives, in particular triptorelin pamoate, somatostatin derivatives, in particular vapreotide pamoate, natural or synthetic heparin, neuroleptics, PTH, insulin and other hypoglycemic peptides, calcitonin, interferons, interleukins, EPO, CSF, oxaliplatin, antidiabetics, such as glizipide, α-reductase inhibitors, thyroxin, estrogens, huperzine and its derivatives,
as polymer, a copolymer of lactic acid and glycolic acid, polylactic acid, a copolymer of polylactic acid and caprolactone, a copolymer of polyethylene glycol or polypropylene glycol with other groups, such as PLGA-PEG coblocks, PLA-PEG or PBS-PEG, polyorthoesters and polyphosphazenes and their copolymers with PEG, and
as solvent, ethyl acetate.

In the process according to the invention, the aqueous phase can comprise 0.05 to 5% of surfactant and at least 0.1 to 2%.

According to the present invention, the surfactants used are polyvinylpyrrolidone, polyvinyl alcohol, carboxymethylcellulose, lecithin or gelatin, anionic surfactants, such as sodium oleate, sodium stearate or sodium lauryl sulfate, or nonionic surfactants, such as polyoxyethylenated sorbitan esters or a polyoxy-ethylenated castor oil derivative.

Preferably, the surfactant used is polyvinyl alcohol.

The examples below are there to illustrate the invention but are not limiting.

The dimensions of the microparticles are measured by laser particle sizing using a device, the Mastersizer® (Malvern Instruments), and the dimensions of the nanoparticles are measured using a device, the Zetasizer® (Malvern Instruments).

A homogenizer, such as the Polytron PT 3000/PT 3100, is used for the preparation of the organic phase.

The degree of encapsulation is measured by an appropriate analytical method, for example by the HPLC-UV method following extraction into triethanolamine phosphate (TEAP) for the peptides or also by UV-visible spectrophotometry after complete dissolution in dimethylformamide (DMF) for olanzapine.

The encapsulation yield, expressed as %, corresponds to the ratio of the degree of encapsulation measured to the theoretical degree of encapsulation.

The description of the present invention is made with reference to the drawings, in which:

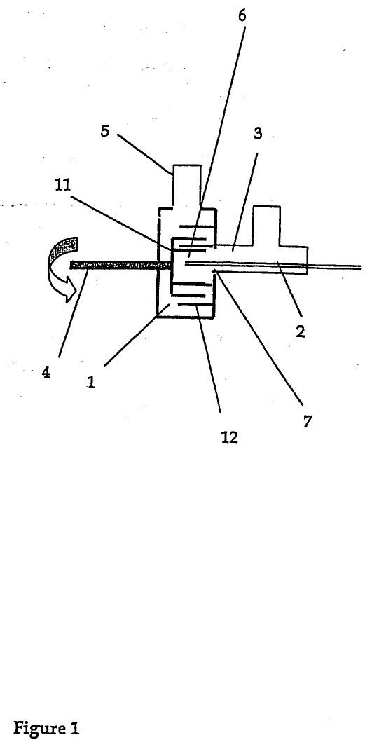

FIG. 1 is a diagrammatic representation of the device according to the present invention for the manufacture of microparticles or nanoparticles,

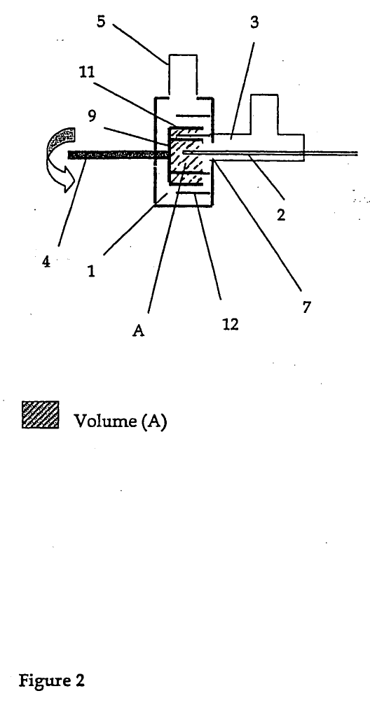

FIG. 2 is a diagrammatic representation of the volume (A) of the device according to the present invention for the manufacture of microparticles or nanoparticles,

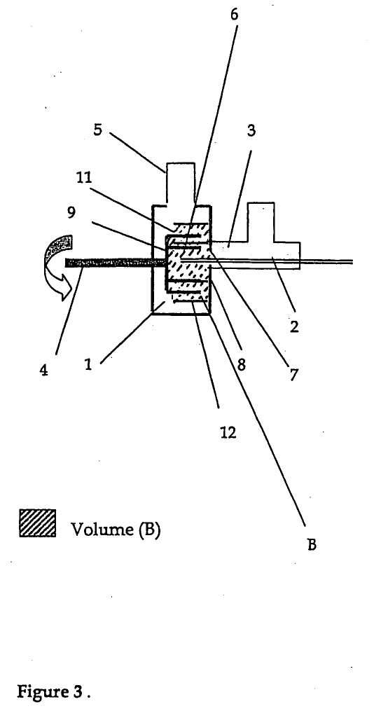

FIG. 3 is a diagrammatic representation of the volume (B) delimited between the wall (8) of the homogenization (1) and the end (9) of the mixing system (4) in the device according to the present invention for the manufacture of microparticles or nanoparticles,

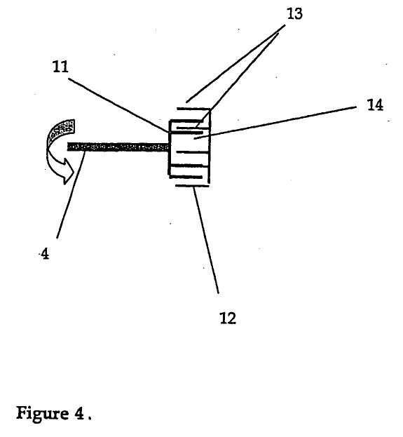

FIG. 4 is a diagrammatic representation of the mixing system (4) consisting of a rotor (11)/stator (12) and present in the homogenization chamber (1),

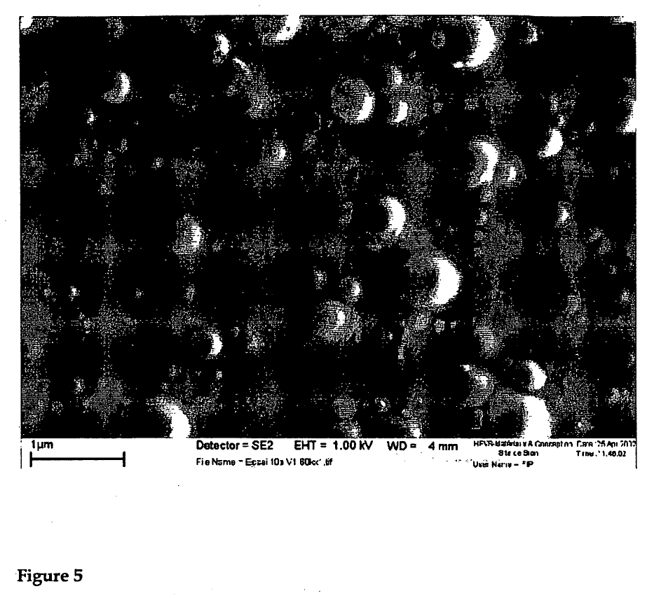

FIG. 5 is a photograph of nanoparticles manufactured according to the use of the process according to the present invention,

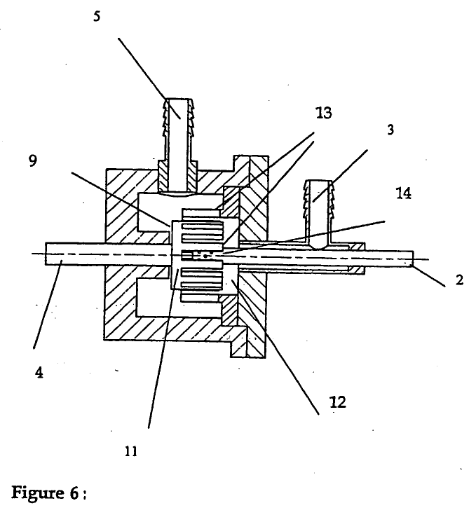

FIG. 6 is also a diagrammatic representation of the mixing system (4) consisting of a rotor (11)/stator (12) and present in the homogenization chamber (1),

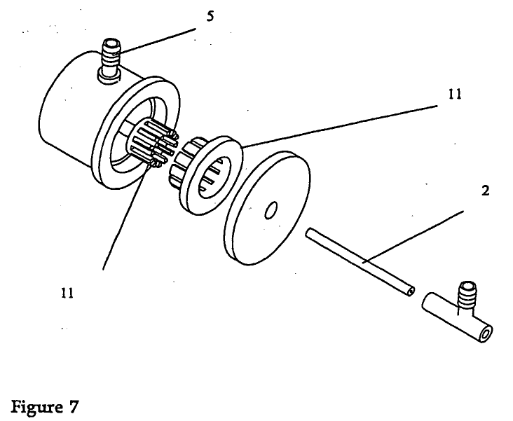

FIG. 7 is a diagrammatic representation of the rotor (11)/stator (12) present in the homogenization chamber (1), of the inlets (2) and (3) and of the outlet (5), and

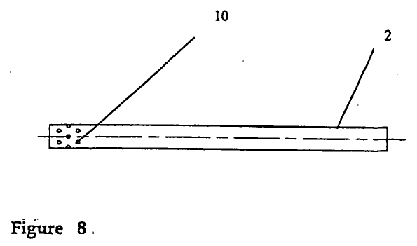

FIG. 8 is a diagrammatic representation of a hollow tube exhibiting perforations (10).

### EXAMPLE 1

Microparticles formed of vapreotide acetate in 50/50 PLGA of low molecular weight are prepared.

For this, the aqueous phase is prepared by mixing 5 g of polyvinyl alcohol and 245 g of water with magnetic stirring at a temperature of 40° C.

At the same time, the organic phase is prepared by completely dissolving 2 g of 50/50 D,L-lactide-co-glycolide (PLGA) polymer in 8 g of ethyl acetate with magnetic stirring. The PLGA polymer exhibits an intrinsic viscosity of 0.17 dl/g, corresponding to an average molecular weight of 10 000.

329 mg of vapreotide acetate are dissolved with magnetic stirring in 800 μl of DMSO (dimethyl sulfoxide) and then this solution is incorporated in the above organic phase. A homogeneous solution (organic phase) is obtained.

The device according to the invention is used.

11.1 g of the organic phase as prepared above are delivered to the homogenization compartment (1) via the inlet (2), the hollow tube, at a flow rate of 10 g/min simultaneously with the aqueous phase as prepared above via the inlet (3) at a flow rate of 150 ml/min.

In order simultaneously to form an emulsion of the two phases and to extract the solvent from the organic phase, the rotor (11)/stator (12) system is rotated at a tangential velocity of 3.2 m/s, i.e. 4000 rpm.

A particle suspension is thus obtained, from which the vapreotide acetate microparticles are isolated by filtration through a 1.2 μm membrane.

The particles are washed with purified water.

Said particles can subsequently be frozen and freeze-dried.

The degree of encapsulation measured is 9.4%, which corresponds to an encapsulation yield of approximately 75%. The mean diameter of the particles is 25 μm.

### EXAMPLE 2

Microparticles formed of vapreotide acetate in 50/50 PLGA with a molecular weight of 35 000 and an intrinsic viscosity of 0.34 dl/g are prepared.

For this, the aqueous phase and the organic phase are prepared as mentioned above in example 1.

The device according to the invention is used.

11.1 g of the organic phase as prepared above are delivered to the homogenization compartment (1) via the inlet (2), the hollow tube, at a flow rate of 10 g/min simultaneously with the aqueous phase as prepared above via the inlet (3) at a flow rate of 150 ml/min.

In order simultaneously to form an emulsion of the two phases and to extract the solvent from the organic phase, the rotor (11)/stator (12) system is rotated at a tangential velocity of 3.2 m/s, i.e. 4000 rpm.

A particle suspension is thus obtained, from which the vapreotide acetate microparticles are isolated by filtration through a 1.2 μm membrane.

The particles are washed with purified water.

Said particles can subsequently be frozen and freeze-dried.

The degree of encapsulation measured is 8.5%, which corresponds to an encapsulation yield of approximately 68%. The mean diameter of the particles is 30 μm.

### EXAMPLE 3

Microparticles formed of vapreotide pamoate in 50/50 PLGA with a molecular weight of 35 000 are prepared.

For this, the aqueous phase is prepared by mixing 5 g of polyvinyl alcohol and 245 g of water with magnetic stirring at a temperature of 40° C.

At the same time, the organic phase is prepared by completely dissolving 4 g of 50/50 poly(D,L-lactide-co-glycolide) (PLGA) polymer in 8 g of ethyl acetate with magnetic stirring.

700 mg of vapreotide pamoate are suspended in 8 g of ethyl acetate using the Polytron homogenizer at 20 000 rpm for 3 minutes, then this suspension is incorporated in the above organic solution and the combined mixture is homogenized using the Polytron homogenizer at 3000 rpm for 20 seconds.

The device according to the invention is used.

20.7 g of the organic phase as prepared above are delivered to the homogenization compartment (1) via the inlet (2), the hollow tube, at a flow rate of 10 g/min simultaneously with the aqueous phase as prepared above via the inlet (3) at a flow rate of 150 ml/min.

In order simultaneously to form an emulsion of the two phases and to extract the solvent from the organic phase, the rotor (11)/stator (12) system is rotated at a tangential velocity of 3.2 m/s, i.e. 4000 rpm.

A particle suspension is thus obtained, from which the vapreotide pamoate microparticles are isolated by filtration through a 1.2 μm membrane.

The particles are washed with purified water.

Said particles can subsequently be frozen and freeze-dried.

The degree of encapsulation measured is 10%, which corresponds to an encapsulation yield of approximately 96%. The mean diameter of the particles is 32 μm.

### EXAMPLE 4

Microparticles formed of olanzapine in 50/50 PLGA with a molecular weight of 35 000 are prepared.

For this, the aqueous phase is prepared by mixing 5 g of polyvinyl alcohol and 245 g of water with magnetic stirring at a temperature of 40° C.

At the same time, the organic phase is prepared by completely dissolving 2 g of 50/50 poly(D,L-lactide-co-glycolide) (PLGA) polymer in 8 g of ethyl acetate with magnetic stirring.

225 mg of olanzapine are dissolved in the organic phase.

The device according to the invention is used.

10.2 g of the organic phase as prepared above are delivered to the homogenization compartment (1) via the inlet (2), the hollow tube, at a flow rate of 10 g/min simultaneously with the aqueous phase as prepared above via the inlet (3) at a flow rate of 150 ml/min.

In order simultaneously to form an emulsion of the two phases and to extract the solvent from the organic phase, the rotor (11)/stator (12) system is rotated at a tangential velocity of 3.2 m/s, i.e. 4000 rpm.

A particle suspension is thus obtained, from which the olanzapine microparticles are isolated by filtration through a 1.2 μm membrane.

The particles are washed with purified water.

Said particles can subsequently be frozen and freeze-dried.

The degree of encapsulation measured is 6.9%, which corresponds to an encapsulation yield of approximately 68%. The mean diameter of the particles is 44 μm.

### EXAMPLE 5

Microparticles formed of triptorelin acetate in 50/50 PLGA with a molecular weight of 35 000 are prepared.

For this, the aqueous phase is prepared by mixing 5 g of polyvinyl alcohol and 245 g of water with magnetic stirring at a temperature of 40° C.

At the same time, the organic phase is prepared by completely dissolving 2 g of 50/50 poly(D,L-lactide-co-glycolide) (PLGA) polymer in 4 g of ethyl acetate with magnetic stirring.

200 mg of triptorelin acetate are suspended in 4 g of ethyl acetate using the Polytron homogenizer at 20 000 rpm and then this suspension is incorporated in the above organic phase. The combined mixture is homogenized with the Polytron homogenizer at 3000 rpm.

The device according to the invention is used.

10.2 g of the organic phase as prepared above are delivered to the homogenization compartment (1) via the inlet (2), which is a hollow tube, at a flow rate of 10 g/min simultaneously with the aqueous phase as prepared above via the inlet (3) at a flow rate of 150 ml/min.

In order simultaneously to form an emulsion of the two phases and to extract the solvent from the organic phase, the rotor (11)/stator (12) system is rotated at a tangential velocity of 3.2 m/s, i.e. 4000 rpm.

A particle suspension is thus obtained, from which the triptorelin acetate microparticles are isolated by filtration through a 1.2 μm membrane.

The particles are washed with purified water.

Said particles can subsequently be frozen and freeze-dried.

The degree of encapsulation measured is 8.9%, which corresponds to an encapsulation yield of approximately 100%. The mean diameter of the particles is 45 μm.

### EXAMPLE 6

Microparticles formed of salmon calcitonin in 50/50 PLGA with an average molecular weight of 35 000 are prepared.

For this, the aqueous phase is prepared by mixing 5 g of polyvinyl alcohol and 245 g of water with magnetic stirring at a temperature of 40° C.

At the same time, the organic phase is prepared by completely dissolving 2 g of 50/50 poly(D,L-lactide-co-glycolide) (PLGA) polymer in 4 g of ethyl acetate with magnetic stirring.

200 mg of salmon calcitonin are suspended in 4 g of ethyl acetate using a Polytron homogenizer at 20 000 rpm and then this suspension is incorporated in the above organic phase. The combined mixture is homogenized using the Polytron homogenizer at 3000 rpm for 20 seconds.

The device according to the invention is used.

11.1 g of the organic phase as prepared above are delivered to the homogenization compartment (1) via the inlet (2), which is a hollow tube, at a flow rate of 10 g/min simultaneously with the aqueous phase as prepared above via the inlet (3) at a flow rate of 150 ml/min.

In order simultaneously to form an emulsion of the two phases and to extract the solvent from the organic phase, the rotor (11)/stator (12) system is rotated at a tangential velocity of 3.2 m/s, i.e. 4000 rpm.

A particle suspension is thus obtained, from which the salmon calcitonin microparticles are isolated by filtration through a 1.2 μm membrane.

The particles are washed with purified water.

Said particles can subsequently be frozen and freeze-dried.

The mean diameter of the particles is 40 μm.

### EXAMPLE 7

Microparticles formed of sodium alendronate in 50/50 PLGA with an average molecular weight of approximately 35 000 are prepared.

For this, the aqueous phase is prepared by mixing 5 g of polyvinyl alcohol and 245 g of water with magnetic stirring at a temperature of 40° C.

At the same time, the organic phase is prepared by completely dissolving 4 g of 50/50 poly(D,L-lactide-co-glycolide) (PLGA) polymer in 8 g of ethyl acetate with magnetic stirring.

200 mg of sodium alendronate are suspended in 8 g of ethyl acetate using a Polytron homogenizer at 20 000 rpm and then this suspension is incorporated in the above organic phase. The combined mixture is homogenized using the Polytron homogenizer at 3000 rpm for 20 seconds.

The device according to the invention is used.

11.1 g of the organic phase as prepared above are delivered to the homogenization compartment (1) via the inlet (2), which is a hollow tube, at a flow rate of

10 g/min simultaneously with the aqueous phase as prepared above via the inlet (3) at a flow rate of 150 ml/min.

In order simultaneously to form an emulsion of the two phases and to extract the solvent from the organic phase, the rotor (11)/stator (12) system is rotated at a tangential velocity of 3.2 m/s, i.e. 4000 rpm.

A particle suspension is thus obtained, from which the sodium alendronate microparticles are isolated by filtration through a 1.2 μm membrane.

The particles are washed with purified water.

Said particles can subsequently be frozen and freeze-dried.

The mean diameter of the particles is 46 μm.

### EXAMPLE 8

Microparticles formed of triptorelin acetate in 50/50 PLGA with a molecular weight of approximately 35 000 are prepared.

For this, the aqueous phase is prepared by mixing 5 g of polyvinyl alcohol and 245 g of water with magnetic stirring at a temperature of 40° C.

At the same time, the organic phase is prepared by completely dissolving 2 g of 50/50 poly(D,L-lactide-co-glycolide) (PLGA) polymer in 8 g of ethyl acetate with magnetic stirring.

100 mg of triptorelin acetate are dissolved in 1.3 g of 20% Tween 80 solution and then this solution is emulsified in the above organic phase using the Polytron homogenizer at 15 000 rpm for 3 minutes.

The device according to the invention is used.

10.1 g of the organic phase as prepared above are delivered to the homogenization compartment (1) via the hollow tube (2) at a flow rate of 10 g/min simultaneously with the aqueous phase as prepared above via the inlet (3) at a flow rate of 150 ml/min.

In order simultaneously to form an emulsion of the two phases and to extract the solvent from the organic phase, the rotor (11)/stator (12) system is rotated at a tangential velocity of 3.2 m/s, i.e. 4000 rpm.

A particle suspension is thus obtained, from which the triptorelin acetate microparticles are isolated by filtration through a 1.2 μm membrane.

The particles are washed with purified water.

Said particles can subsequently be frozen and freeze-dried.

The degree of encapsulation measured is 5.8%, which corresponds to an encapsulation yield of approximately 100%. The mean diameter of the particles is 19 μm.

### EXAMPLE 9

Microparticles formed of triptorelin pamoate in 85/15 PLGA with an average molecular weight of approximately 74 000 are prepared.

For this, the aqueous phase is prepared by mixing 100 g of polyvinyl alcohol and 4900 g of water with magnetic stirring at a temperature of 40° C.

At the same time, the organic phase is prepared by completely dissolving 4 g of 85/15 poly(D,L-lactide-co-glycolide) (PLGA) polymer in 15 g of dichloromethane with magnetic stirring.

1000 mg of triptorelin pamoate are suspended in 10 g of dichloromethane with magnetic stirring and then this solution is incorporated in the above organic phase.

The device according to the invention is used.

30 g of the organic phase as prepared above are delivered to the homogenization compartment (1) via the hollow tube (2) at a flow rate of 5 ml/min simultaneously with the aqueous phase as prepared above via the inlet (3) at a flow rate of 850 ml/min.

In order simultaneously to form an emulsion of the two phases and to extract the solvent from the organic phase, the rotor (11)/stator (12) system is rotated at a tangential velocity of 4.4 m/s, i.e. 5500 rpm.

A particle suspension is thus obtained, from which the triptorelin pamoate microparticles are isolated by filtration through a 1.2 μm membrane.

The particles are washed with purified water.

Said particles can subsequently be frozen and freeze-dried.

The degree of encapsulation measured is 12.54%, which corresponds to an encapsulation yield of approximately 90%. The mean diameter of the particles is 70 μm.

### EXAMPLE 10

Nanoparticles formed of olanzapine in 50/50 PLGA with a molecular weight of 35 000 are prepared.

For this, the aqueous phase is prepared by mixing 5 g of polyvinyl alcohol and 245 g of water with magnetic stirring at a temperature of 40° C.

At the same time, the organic phase is prepared by completely dissolving 2 g of 50/50 poly(D,L-lactide-co-glycolide) (PLGA) polymer in 8 g of ethyl acetate with magnetic stirring.

225 mg of olanzapine are then dissolved in the above organic phase.

The device according to the invention is used.

10.2 g of the organic phase as prepared above are delivered to the homogenization compartment (1) via the hollow tube (2) at a flow rate of 10 g/min simultaneously with the aqueous phase as prepared above via the inlet (3) at a flow rate of 200 ml/min.

In order simultaneously to form an emulsion of the two phases and to extract the solvent from the organic phase, the rotor (11)/stator (12) system is rotated at a tangential velocity of 40 m/s, i.e. 30 000 rpm.

A particle suspension is thus obtained, from which the olanzapine nanoparticles are isolated by centrifuging and filtration.

Said particles can subsequently be frozen and freeze-dried.

The degree of encapsulation measured is 3.3%, which corresponds to an encapsulation yield of approximately 33%. The mean of the measured particles is 230 nm.

### EXAMPLE 11

Nanoparticles formed of olanzapine in PBS-PEG with a molecular weight of approximately 30 000 are prepared.

For this, the aqueous phase is prepared by mixing 40 g of polyvinyl alcohol and 1200 g of water with magnetic stirring at a temperature of 40° C.

At the same time, the organic phase is prepared by completely dissolving 0.9 g of PBS-PEG polymer with a viscosity of 0.64 dl/g (as prepared in example 10 of patent application WO 99/55760) and 100 mg of olanzapine in 19 g of dichloromethane with magnetic stirring.

The device according to the invention is used.

22 g of the organic phase as prepared above are delivered to the homogenization compartment (1) via the hollow tube (2) at a flow rate of 10.9 ml/min simultaneously with the aqueous phase as prepared above via the inlet (3) at a flow rate of 400 ml/min.

In order simultaneously to form an emulsion of the two phases and to extract the solvent from the organic phase, the rotor (11)/stator (12) system is rotated at a tangential velocity of 40 m/s, i.e. 30 000 rpm.

A particle suspension is thus obtained, from which the olanzapine nanoparticles are isolated by filtration through a 0.22 μm filter.

The degree of encapsulation measured is 3%, which corresponds to an encapsulation yield of approximately 30%. The mean of the measured particles is 50 nm.

### EXAMPLE 12

Microparticles formed of triptorelin pamoate in 85/15 PLGA with a molecular weight of 74 000 are prepared.

For this, the aqueous phase is prepared by mixing 100 g of polyvinyl alcohol and 4900 g of water with magnetic stirring at a temperature of 40° C.

At the same time, the organic phase is prepared by completely dissolving 4 g of 85/15 poly(D,L-lactide-co-glycolide) (PLGA) polymer in 15 g of ethyl acetate with magnetic stirring.

1000 mg of triptorelin pamoate are dispersed in 10 g of ethyl acetate using the Polytron homogenizer (20 000 rpm, 6 minutes) and then this suspension is incorporated in the above organic phase.

The device according to the invention is used.

The organic phase as prepared above is delivered to the homogenization compartment (1) via the hollow tube (2) at a flow rate of 5 ml/min simultaneously with the aqueous phase as prepared above via the inlet (3) at a flow rate of 200 ml/min.

In order simultaneously to form an emulsion of the two phases and to extract the solvent from the organic phase, the rotor (11)/stator (12) system is rotated at a tangential velocity of 4.4 m/s, i.e. 5500 rpm.

A particle suspension is thus obtained, from which the triptorelin pamoate microparticles are isolated by filtration through a 1.2 μm membrane.

The particles are washed with purified water.

Said particles can subsequently be frozen and freeze-dried.

The degree of encapsulation measured is 11.27%, which corresponds to an encapsulation yield of approximately 80%. The mean diameter of the particles is 33.9 μm.

### EXAMPLE 13

Microparticles formed of triptorelin pamoate in 85/15 PLGA with a molecular weight of 74 000 are prepared.

For this, the aqueous phase is prepared by mixing 10 g of polyvinyl alcohol and 1990 g of water with magnetic stirring at a temperature of 40° C.

At the same time, the organic phase is prepared by completely dissolving 4 g of 85/15 poly(D,L-lactide-co-glycolide) (PLGA) polymer in 15 g of ethyl acetate with magnetic stirring.

1000 mg of triptorelin pamoate are suspended in 10 g of ethyl acetate using the Polytron homogenizer (20 000 rpm, 6 minutes) and then this suspension is incorporated in the above organic phase.

The device according to the invention is used.

The organic phase as prepared above is delivered to the homogenization compartment (1) via the hollow tube (2) at a flow rate of 5 ml/min simultaneously with the aqueous phase as prepared above via the inlet (3) at a flow rate of 200 ml/min.

In order simultaneously to form an emulsion of the two phases and to extract the solvent from the organic phase, the rotor (11)/stator (12) system is rotated at a tangential velocity of 4.4 m/s, i.e. 5500 rpm.

A particle suspension is thus obtained, from which the triptorelin pamoate microparticles are isolated by filtration through a 1.2 μm membrane.

The particles are washed with purified water.

Said particles can subsequently be frozen and freeze-dried.

The degree of encapsulation measured is 12.4%, which corresponds to an encapsulation yield of approximately 89%. The mean diameter of the particles is 46.2 μm.

### EXAMPLE 14

Microparticles formed of triptorelin pamoate in 90/10 PLGA with a molecular weight of approximately 30 000 are prepared.

For this, the aqueous phase is prepared by mixing 40 g of polyvinyl alcohol and 1960 g of purified water with magnetic stirring at a temperature of 40° C.

At the same time, the organic phase is prepared by completely dissolving 4 g of 90/10 poly(D,L-lactide-co-glycolide) (PLGA) polymer in 15 g of ethyl acetate with magnetic stirring.

1000 mg of triptorelin pamoate are suspended in 10 g of ethyl acetate using the Polytron homogenizer (20 000 rpm, 6 minutes) and then this solution is incorporated in the above organic phase.

The device according to the invention is used.

The organic phase as prepared above is delivered to the homogenization compartment (1) via the hollow tube (2) at a flow rate of 5 ml/min simultaneously with the aqueous phase as prepared above via the inlet (3) at a flow rate of 200 ml/min.

In order simultaneously to form an emulsion of the two phases and to extract the solvent from the organic phase, the rotor (11)/stator (12) system is rotated at a tangential velocity of 4.4 m/s, i.e. 5500 rpm.

A particle suspension is thus obtained, from which the triptorelin pamoate microparticles are isolated by filtration through a 1.2 μm membrane.

The particles are washed with purified water.

Said particles can subsequently be frozen and freeze-dried.

The degree of encapsulation measured is 10.5%, which corresponds to an encapsulation yield of approximately 75%. The mean diameter of the particles is 20.7 μm.

### EXAMPLE 15

Microparticles formed of triptorelin pamoate in PLA with a molecular weight of approximately 30 000 are prepared.

For this, the aqueous phase is prepared by mixing 40 g of polyvinyl alcohol and 1960 g of water with magnetic stirring at a temperature of 40° C.

At the same time, the organic phase is prepared by completely dissolving 4 g of poly(D,L-lactide) (PLA) polymer in 15 g of ethyl acetate with magnetic stirring.

1000 mg of triptorelin pamoate are suspended in 10 g of ethyl acetate using the Polytron homogenizer (20 000 rpm, 6 minutes) and then this solution is incorporated in the above organic phase.

The device according to the invention is used.

The organic phase as prepared above is delivered to the homogenization compartment (1) via the hollow tube (2) at a flow rate of 5 ml/min simultaneously with the aqueous phase as prepared above via the inlet (3) at a flow rate of 200 ml/min.

In order simultaneously to form an emulsion of the two phases and to extract the solvent from the organic phase, the rotor (11)/stator (12) system is rotated at a tangential velocity of 4.4 m/s, i.e. 5500 rpm.

A particle suspension is thus obtained, from which the triptorelin pamoate microparticles are isolated by filtration through a 1.2 μm membrane.

The particles are washed with purified water.

Said particles can subsequently be frozen and freeze-dried.

The degree of encapsulation measured is 10%, which corresponds to an encapsulation yield of approximately 71%. The mean diameter of the particles is 21.6 μm.

### EXAMPLE 16

Microparticles formed of triptorelin pamoate in PLA with a molecular weight of approximately 70 000 are prepared.

For this, the aqueous phase is prepared by mixing 40 g of polyvinyl alcohol and 1960 g of water with magnetic stirring at a temperature of 40° C.

At the same time, the organic phase is prepared by completely dissolving 4 g of poly(D,L-lactide) (PLA) polymer in 15 g of ethyl acetate with magnetic stirring.

1000 mg of triptorelin pamoate are suspended in 10 g of ethyl acetate using the Polytron homogenizer (20 000 rpm, 6 minutes) and then this suspension is incorporated in the above organic phase.

The device according to the invention is used.

The organic phase as prepared above is delivered to the homogenization compartment (1) via the hollow tube (2) at a flow rate of 5 ml/min simultaneously with the aqueous phase as prepared above via the inlet (3) at a flow rate of 200 ml/min.

In order simultaneously to form an emulsion of the two phases and to extract the solvent from the organic phase, the rotor (11)/stator (12) system is rotated at a tangential velocity of 4.4 m/s, i.e. 5500 rpm.

A particle suspension is thus obtained, from which the triptorelin pamoate microparticles are isolated by filtration through a 1.2 μm membrane.

The particles are washed with purified water.

Said particles can subsequently be frozen and freeze-dried.

The degree of encapsulation measured is 11.5%, which corresponds to an encapsulation yield of approximately 82%. The mean diameter of the particles is 32.1 μm.

### EXAMPLE 17

Microparticles formed of triptorelin pamoate in PLA with a molecular weight of approximately 20 000 are prepared.

For this, the aqueous phase is prepared by mixing 40 g of polyvinyl alcohol and 1960 g of water with magnetic stirring at a temperature of 40° C.

At the same time, the organic phase is prepared by completely dissolving 4 g of poly(D,L-lactide) (PLA) polymer in 15 g of ethyl acetate with magnetic stirring.

1000 mg of triptorelin pamoate are suspended in 10 g of ethyl acetate using the Polytron homogenizer (20 000 rpm, 6 minutes) and then this suspension is incorporated in the above organic phase.

The device according to the invention is used.

The organic phase as prepared above is delivered to the homogenization compartment (1) via the hollow tube (2) at a flow rate of 5 ml/min simultaneously with the aqueous phase as prepared above via the inlet (3) at a flow rate of 200 ml/min.

In order simultaneously to form an emulsion of the two phases and to extract the solvent from the organic phase, the rotor (11)/stator (12) system is rotated at a tangential velocity of 4.4 m/s, i.e. 5500 rpm.

A particle suspension is thus obtained, from which the triptorelin pamoate microparticles are isolated by filtration through a 1.2 μm membrane.

The particles are washed with purified water.

Said particles can subsequently be frozen and freeze-dried.

The degree of encapsulation measured is 8.83%, which corresponds to an encapsulation yield of approximately 63%. The mean diameter of the particles is 22.2 μm.

### EXAMPLE 18

Microparticles formed of triptorelin pamoate in a 75/25 poly(D,L-lactide-co-ε-caprolactone) (PLA-PCL) copolymer with a molecular weight of 80 000 are prepared.

For this, the aqueous phase is prepared by mixing 40 g of polyvinyl alcohol and 1960 g of water with magnetic stirring at a temperature of 40° C.

At the same time, the organic phase is prepared by completely dissolving 4 g of 75/25 poly(D,L-lactide-co-ε-caprolactone) (PLA-PCL) copolymer in 15 g of ethyl acetate with magnetic stirring.

1000 mg of triptorelin pamoate are suspended in 10 g of ethyl acetate and then this suspension is incorporated in the above organic phase.

The device according to the invention is used.

The organic phase as prepared above is delivered to the homogenization compartment (1) via the hollow tube (2) at a flow rate of 5 ml/min simultaneously with the aqueous phase as prepared above via the inlet (3) at a flow rate of 200 ml/min.

In order simultaneously to form an emulsion of the two phases and to extract the solvent from the organic phase, the rotor (11)/stator (12) system is rotated at a tangential velocity of 4.4 m/s, i.e. 5500 rpm.

A particle suspension is thus obtained, from which the triptorelin pamoate microparticles are isolated by filtration through a 1.2 μm membrane.

The particles are washed with purified water.

Said particles can subsequently be frozen and freeze-dried.

The mean diameter of the particles is 35.2 μm.

### EXAMPLE 19

Microparticles formed of heparin of low molecular weight are prepared.

For this, the aqueous phase is prepared by mixing 260 g of polyvinyl alcohol and 12 740 g of MilliQ H₂O with magnetic stirring at a temperature of 40° C.

At the same time, the organic phase is prepared by completely dissolving 4.26 g of a mixture comprising 50% of 50/50 poly(D,L-lactide-co-glycolide) (PLGA) polymer with a viscosity of 0.5 dl/g and 50% of Eudragit RS PO polymer in 83.5 g of ethyl acetate with magnetic stirring.

750 mg of nadroparin are suspended in 16.7 ml of purified water and then this solution is emulsified in the organic phase using the Polytron homogenizer (15 000 rpm, 90 seconds).

The device according to the invention is used.

The organic phase as prepared above is delivered to the homogenization compartment (1) via the inlet (2), the hollow tube, at a flow rate of 5 ml/min simultaneously with the aqueous phase as prepared above via the inlet (3) at a flow rate of 650 ml/min.

In order simultaneously to form an emulsion of the two phases and to extract the solvent from the organic phase, the rotor (11)/stator (12) system is rotated at a tangential velocity of 3.2 m/s, i.e. 4000 rpm.

A particle suspension is thus obtained, from which the particles are isolated by filtration through a 1.2 μm membrane.

The particles are washed with purified water.

Said particles can subsequently be frozen and freeze-dried.

The mean diameter of the microparticles is 23 μm.

### EXAMPLE 20

Microparticles formed of interferon are prepared.

For this, the aqueous phase is prepared by mixing 120 g of polyvinyl alcohol and 5880 g of MilliQ H₂O with magnetic stirring at a temperature of 40° C.

At the same time, the organic phase is prepared by completely dissolving 1.98 g of a mixture comprising 50% of 50/50 poly(D,L-lactide-co-glycolide) (PLGA) polymer with a viscosity of 0.5 dl/g and 50% of Eudragit RS PO polymer in 39 ml of ethyl acetate with magnetic stirring.

7 ml of the interferon solution are prepared by mixing 381 μl of the solution of interferon α-17 in phosphate buffer pH 8 (1.83 mg of protein/ml), 280 μl of the solution of human serum albumin (50 mg/ml) in phosphate buffer pH 8 and 6939 μl of phosphate buffer pH 8.

The interferon solution thus prepared is emulsified in the organic phase using a Polytron homogenizer (15 000 rpm, 90 seconds).

The device according to the invention is used.

The organic phase as prepared above is delivered to the homogenization compartment (1) via the inlet (2), the hollow tube, at a flow rate of 5 ml/min simultaneously with the aqueous phase as prepared above via the inlet (3) at a flow rate of 590 ml/min.

In order simultaneously to form an emulsion of the two phases and to extract the solvent from the organic phase, the rotor (11)/stator (12) system is rotated at a tangential velocity of 2.4 m/s, i.e. 3000 rpm.

A particle suspension is thus obtained, from which the particles are isolated by filtration through a 1.2 μm membrane.

The particles are washed with purified water.

Said particles can subsequently be freeze-dried.

The mean diameter of the microparticles is 19.1 μm.

### COUNTEREXAMPLE 21

Use is made of the process and of a homogenizer of Silverson type as are disclosed in EP 0471 036.

An aqueous phase is prepared by mixing, with magnetic stirring, 10 g of PVA (polyvinyl alcohol), 0.847 g of anhydrous sodium hydrogenphosphate and 489 g of MilliQ H₂O. Finally, 39 g of ethyl acetate are added so as to stabilize the pH at 8.9.

At the same time, an organic phase is prepared by dissolving 3.4 g of 50/50 poly(D,L-lactide-co-glycolide) (PLGA) polymer with a viscosity of 0.34 dl/g in 3.4 g of ethyl acetate with magnetic stirring.

571 mg of leuprolide acetate are also dissolved in 1.549 g of DMSO.

This solution comprising the leuprolide acetate is mixed into the organic phase with magnetic stirring.

The organic phase thus prepared is pumped into the homogenizer of Silverson type equipped with a 4-bladed rotor rotating at 400 rpm.

At the same time, the aqueous phase is also pumped into this homogenizer at a rate of 127 ml/min.

A first emulsion, referred to as the primary emulsion, is thus obtained. A portion of the solvent present in this primary emulsion is extracted at the outlet of the Silverson homogenizer by pumping purified water at a flow rate of 1790 ml/min.

A suspension of microspheres is thus obtained and is collected in a receptacle containing 500 ml of purified water in which said suspension is left under magnetic stirring for 15 min, so as to extract the remaining solvent present in this suspension.

Finally, the particle suspension is filtered, so as to obtain separate particles which are freeze-dried.

The particles thus obtained are elongated and nonhomogeneous in shape and sieving through a 106 μm mesh is difficult.

### EXAMPLE 22

Nanoparticles formed of estradiol valerate in 50/50 PLGA with a molecular weight of 35 000 are prepared.

For this, the aqueous phase is prepared by mixing 5 g of polyvinyl alcohol and 245 g of water with magnetic stirring at ambient temperature.

At the same time, the organic phase is prepared by completely dissolving 2.467 g of 50/50 poly(D,L-lactide-co-glycolide) (PLGA) polymer in 50 ml of ethyl acetate with magnetic stirring.

33 mg of estradiol valerate are then dissolved in the above organic phase.

The device according to the invention is used.

All of the organic phase as prepared above is delivered to the homogenization compartment (1) via the hollow tube (2) at a flow rate of 5 ml/min simultaneously with the aqueous phase as prepared above via the inlet (3) at a flow rate of 10 ml/min, until the organic phase has been used up.

In order simultaneously to form an emulsion of the two phases and to extract the solvent from the organic phase, the rotor (11)/stator (12) system is rotated at a tangential velocity of 26.6 m/s, i.e. 20 000 rpm.

A suspension of estradiol valerate nanoparticles with a measured mean size of 300 nm is thus obtained.

Said particles can subsequently be rozen and freeze-dried.

### COUNTERXAMPLE 23

Use is made of the process and of a homogenizer of Silverson type as are disclosed in patent WO 03/033097. An attempt is made to prepare microparticles formed of estradiol valerate in 75/25 PLGA.

For this, the aqueous phase is prepared by mixing 100 g of polyvinyl alcohol and 4900 g of water (2%) with magnetic stirring at ambient temperature.

At the same time, the organic phase is prepared by completely dissolving 2.27 g of 75/25 poly(D,L-lactide-co-glycolide) (PLGA) polymer in 25 g of ethyl acetate with magnetic stirring.

229 mg of estradiol valerate are then dissolved in the above organic phase.

The device according to the invention is used.

The organic phase thus prepared is pumped at a flow rate of 5 ml/min into the homogenizer of Silverson type equipped with a 4-bladed rotor according to patent WO 03/033097 rotating at 5500 rpm.

At the same time, the aqueous phase is also pumped into this homogenizer at a rate of 750 ml/min.

A suspension of microspheres is thus obtained and is collected in a receptacle with magnetic stirring.

By optical microscopy, the suspension comprises microspheres which are nonhomogeneous in size and also filaments.

Finally, the suspension composed of said microparticles and said filaments is filtered through a 1.2 μm filter and then freeze-dried.

The particles obtained are stringy and nonhomogeneous in shape. Suspending is difficult.

### EXAMPLE 24

In this example, use will be made of a hollow tube covered at its end with a multiperforated screen.

Microparticles formed of olanzapine in 50/50 PLGA of low molecular weight are prepared.

For this, the aqueous phase is prepared by mixing 80 g of polyvinyl alcohol and 3920 g of water with magnetic stirring at a temperature of 40° C.

At the same time, the organic phase is prepared by completely dissolving 4.5 g of 50/50 D,L-lactide-co-glycolide (PLGA) polymer in 25 g of ethyl acetate with magnetic stirring. The PLGA polymer exhibits an intrinsic viscosity of 0.34 dl/g, corresponding to an average molecular weight of 35 000 Da.

500 mg of olanzapine are dissolved with magnetic stirring in the above organic phase. A homogeneous solution (organic phase) is obtained.

The device according to the invention is used. The hollow tube employed is covered at its end with a multiperforated screen.

30 g of the organic phase as prepared above are delivered to the homogenization compartment (1) via the hollow tube (2) at a flow rate of 5 ml/min simultaneously with the aqueous phase as prepared above via the inlet (3) at a flow rate of 600 ml/min.

In order simultaneously to form an emulsion of the two phases and to extract the solvent from the organic phase, the rotor (11)/stator (12) system is rotated at a tangential velocity of 4.0 m/s, i.e. 5000 rpm.

A particle suspension is thus obtained, from which the olanzapine microparticles are isolated by filtration through a 1.2 μm membrane.

The particles are washed with purified water.

Said particles can subsequently be frozen and freeze-dried.

The degree of encapsulation measured, is 8.6%, which corresponds to an encapsulation yield of approximately 86%. The mean diameter of the particles is 32 μm.

### EXAMPLE 25

Microparticles formed of estradiol in 50/50 PLGA with a molecular weight of 40 000 Da and an intrinsic viscosity of 0.42 dl/g are prepared.

For this, the aqueous phase is prepared by mixing 160 g of polyvinyl alcohol and 7840 g of water with magnetic stirring at a temperature of 40° C.

At the same time, the organic phase is prepared by completely dissolving 4.9 g of 50/50 D,L-lactide-co-glycolide (PLGA) polymer in 50 g of ethyl acetate with magnetic stirring.

100 mg of estradiol are dissolved with magnetic stirring in 800 μl of DMSO (dimethyl sulfoxide) and then this solution is incorporated in the above organic phase. A homogeneous solution (organic phase) is obtained.

The device according to the invention is used.

55 g of the organic phase as prepared above are delivered to the homogenization compartment (1) via the hollow tube (2) at a flow rate of 5 ml/min simultaneously with the aqueous phase as prepared above via the inlet (3) at a flow rate of 750 ml/min.

In order simultaneously to form an emulsion of the two phases and to extract the solvent from the organic phase, the rotor (11)/stator (12) system is rotated at a tangential velocity of 4.0 m/s, i.e. 5000 rpm.

A particle suspension is thus obtained, from which the estradiol microparticles are isolated by filtration through a 1.2 μm membrane.

The particles are washed with purified water.

Said particles can subsequently be frozen and freeze-dried.

The degree of encapsulation measured is 1.5%, which corresponds to an encapsulation yield of approximately 75%. The mean diameter of the particles is 18.9 μm.

### EXAMPLE 26

Microparticles formed of estradiol in 50/50 PLGA with a molecular weight of 50 000 Da and an intrinsic viscosity of 0.5 dl/g are prepared.

For this, the aqueous phase is prepared by mixing 160 g of polyvinyl alcohol and 7840 g of water with magnetic stirring at a temperature of 40° C.

At the same time, the organic phase is prepared by completely dissolving 4.9 g of 50/50 D,L-lactide-co-glycolide (PLGA) polymer in 50 g of ethyl acetate with magnetic stirring.

100 mg of estradiol are dissolved with magnetic stirring in 800 μl of DMSO (dimethyl sulfoxide) and then this solution is incorporated in the above organic phase. A homogeneous solution (organic phase) is obtained.

The device according to the invention is used.

55 g of the organic phase as prepared above are delivered to the homogenization compartment (1) via the hollow tube (2) at a flow rate of 5 ml/min simultaneously with the aqueous phase as prepared above via the inlet (3) at a flow rate of 750 ml/min.

In order simultaneously to form an emulsion of the two phases and to extract the solvent from the organic phase, the rotor (11)/stator (12) system is rotated at a tangential velocity of 4.0 m/s, i.e. 5000 rpm.

A particle suspension is thus obtained, from which the estradiol microparticles are isolated by filtration through a 1.2 μm membrane.

The particles are washed with purified water.

Said particles can subsequently be frozen and freeze-dried.

The degree of encapsulation measured is 1.7%, which corresponds to an encapsulation yield of approximately 85%. The mean diameter of the particles is 23.6 μm.

### EXAMPLE 27

Microparticles formed of estradiol in 75/25 PLGA with a molecular weight of 70 000 Da and an intrinsic viscosity of 0.65 dl/g are prepared.

For this, the aqueous phase is prepared by mixing 160 g of polyvinyl alcohol and 7840 g of water with magnetic stirring at a temperature of 40° C.

At the same time, the organic phase is prepared by completely dissolving 4.65 g of 75/25 D,L-lactide-co-glycolide (PLGA) polymer in 50 g of ethyl acetate with magnetic stirring.

350 mg of estradiol are dissolved with magnetic stirring in 2500 μl of DMSO (dimethyl sulfoxide) and then this solution is incorporated in the above organic phase. A homogeneous solution (organic phase) is obtained.

The device according to the invention is used.

57 g of the organic phase as prepared above are delivered to the homogenization compartment (1) via the hollow tube (2) at a flow rate of 5 ml/min simultaneously with the aqueous phase as prepared above via the inlet (3) at a flow rate of 750 ml/min.

In order simultaneously to form an emulsion of the two phases and to extract the solvent from the organic phase, the rotor (11)/stator (12) system is rotated at a tangential velocity of 4.4 m/s, i.e. 5500 rpm.

A particle suspension is thus obtained, from which the estradiol microparticles are isolated by filtration through a 1.2 μm membrane.

The particles are washed with purified water.

Said particles can subsequently be frozen and freeze-dried.

The degree of encapsulation measured is 6%, which corresponds to an encapsulation yield of approximately 86%. The mean diameter of the particles is 18.4 μm.

1. A device for the continuous manufacture of microparticles or nanoparticles from at least one aqueous phase and one organic phase comprising a homogenization compartment in the form of a cylinder (1) which is defined by a tubular wall forming the casing of said cylinder and by a first side wall and a second side wall which are positioned at each end of said tubular wall; the device additionally comprising a first inlet and a second inlet (2, 3) which pass through said first side wall and which are appropriate for respectively delivering an organic phase and an aqueous phase to the homogenization compartment (1) and an outlet (5) appropriate for extracting a particle suspension from the homogenization compartment (1); the homogenization compartment (1) including a mixing system (4) comprising a rotor (11)/stator (12) combination, characterized in that
a) said side walls are positioned along a vertical plane,
b) the axis of symmetry of said cylinder is positioned horizontally,
c) the rotor (11) is installed so that it rotates about a horizontal axis which passes through said second side wall,
d) said first inlet (2) is a hollow tube positioned in the extension of the axis of the rotor (11) and comprises a final part (6) situated inside the rotor (11) and inside the stator (12),
e) the homogenization compartment (1) exhibits a top side on which said outlet (5) is situated.

2. The device as claimed in claim 1, characterized in that the rotor (11) and the stator (12) are cylindrical in shape.

3. The device as claimed in claim 5, characterized in that the rotor (11) and the stator (12) comprise a row of teeth (13) and that the spacing (14) between the teeth (13) is from 1 to 4 mm.

4. The device as claimed in claim 1, characterized in that the first inlet (2) comprises perforations (10).

5. The device as claimed in claim 4, characterized in that the number of perforations (10) is from 1 to 20.

6. The device as claimed in claim 4, characterized in that the perforations (10) have a diameter from 0.01 mm to 1 mm.

7. The device as claimed in claim 1, characterized in that the dimensions of the rotor (11 )/stator (12) combination are such that said system occupies 4% to 40% of the volume of the homogenization compartment (1).

8. A continuous process for the manufacture of microparticles or nanoparticles employing the device as claimed in claim 1, characterized in that an organic phase comprising an active subs polymer and a solvent and an aqueous phase comprising a surfactant are simultaneously delivered to the homogenization compartment (1) via the first inlet (2) and via the second inlet (3) respectively and that a tangential velocity of 1.5 m/s to 50 m/s is applied to the rotor (11 )/stator (12) combination so as simultaneously to form an emulsion of said phases and to extract solvent present in the organic phase so as to obtain a particle suspension from which said particles are isolated.

9. The process as claimed in claim 8, characterized in that the organic phase is dispersed radially through perforations (10) positioned on the second inlet (2).

10. The process as claimed in claim 8, in which said particles are isolated by discharging said suspension via the outlet (5) of the homogenization compartment (1) into a storage receptacle and by subjecting said suspension to continuous ultrafiltration.

11. The process as claimed in claim 8, in which said particles are isolated by discharging said suspension via the outlet (5) of the homogenization compartment (1) into a storage receptacle and by subjecting said suspension to continuous filtration.

12. The process for the continuous manufacture of microparticles or nanoparticles from at least one aqueous phase and one organic phase, said process comprising the introduction of an organic phase and of an aqueous phase into a homogenization compartment (1) which includes a mixing system (4) comprising a rotor (11 )/stator (12) combination and then the extraction of a suspension of microparticles or nanoparticles through an outlet situated on a side of the homogenization compartment (1);
characterized in that
a) said introduction of the aqueous phase and of the organic phase is carried out horizontally,
b) the rotor (11 )/stator (12) combination is driven about a horizontal axis,
c) the organic phase is introduced into a space which is situated inside the rotor (11 ) and inside the stator (12),
d) said suspension is extracted through the top side of the homogenization compartment (1).
